(19) United States
(12) Patent Application Publication
(10) Pub. No.: US 2005/0142551 A1
(43) Pub. Date: Jun. 30, 2005

(54) Fabrication of a high resolution biological molecule detection device with aluminum electrical conductors

(76) Inventors: Hong-An Pham, West Henrietta, NY (US); Dennis M. Connolly, Rochester, NY (US)

Correspondence Address: Michael L Goldman;Nixon Peabody, Clinton Square, PO Box 31051, Rochester, NY, 14603, US

(21) Appl. No.: 10/496,323

(22) PCT Filed: Nov. 19, 2002
(86) PCT No.: PCT/US02/37257

Related U.S. Application Data
(60) Provisional application No. 60/332,282, filed on Nov. 21, 2001.

(51) Int. Cl. C12Q 1/68; C12M 1/34; H01L 21/00

(52) U.S. Cl. 435/6; 435/287.2; 438/1

(57) ABSTRACT

The present invention relates to a method of manufacturing a detection device which involves providing a substrate having a layer of aluminum and a first layer of photosensitive material. Next, the substrate is subjected to a first level photolithography treatment to produce an aluminum electrical conductor containing conductive fingers with spaces between them. Finally, biological probes are attached to the conductive fingers under conditions effective to form a gap between the biological probes on the spaced apart conductive fingers. As a result, a target molecule, if present in a sample, can bind to a pair of the biological probes on the spaced apart conductive fingers to bridge the gap between the biological probes, allowing detection of the target molecule.

This application claims the benefit of U.S. Provisional Patent Application Ser. No. 60/332,282, filed Nov. 21, 2001, which is hereby incorporated by reference in its entirety.

### FIELD OF THE INVENTION

The present invention relates to methods of fabricating a device for the detection of target biological molecules from samples.

### BACKGROUND OF THE INVENTION

For the analysis and testing of nucleic acid molecules, amplification of a small amount of nucleic acid molecules, isolation of the amplified nucleic acid fragments, and other procedures are necessary. The polymerase chain reaction method is widely used for the amplification of nucleic acid molecules, in which an extremely small number of nucleic acid molecules or fragments can be multiplied by several orders of magnitude to provide detectable amounts of material. On the other hand, isolation and detection of particular nucleic acid molecules in a mixture requires a nucleic acid sequencer and fragment analyzer, in which gel electrophoresis and fluorescence detection are combined. However, electrophoresis becomes very labor-intensive as the number of samples or test items increases. For this reason, a simpler method of analysis using DNA oligonucleotide probes is becoming popular. In this method, many kinds of oligonucleotide probes are immobilized on the surface of a solid to make a probe array. When contacted with a sample, only nucleic acid molecules with specific sequences matching the oligonucleotide are trapped on the surface of the solid and detected.

This kind of isolation and detection method, in which biological probes are immobilized on the surface of a solid and hybridization proceeds between the probes and a sample, has long been known as a blotting method in which the presence of the target molecule is detected by a probe immobilized on a membrane using radioactive labeling. However, immobilization of a large number of probes on a small area has the advantage that only a small amount of sample is required, and a large number of probes can be used simultaneously.

There are several methods for production of such products. Probe molecules can be synthesized one base at a time by a photochemical reaction on small segments of a solid using the same photomasking techniques used in the semiconductor industry. In another method, a synthesized DNA, a PCR-amplified DNA, or a protein molecule is immobilized on a small segment of the surface of a solid for each probe. A third method is to use an inkjet droplet to deposit the biological probe onto the surface. After the biological probes are attached to the surface, the sample containing the target molecule to be analyzed is passed over the biological probes at a temperature conducive to rapid hybridization of the target molecule with the probes. A washing solution then removes all the unhybridized, unbound molecules.

This method requires the use of fluorescent or radioactive labels as additional materials. Such a system is expensive to use and is not amenable to being made portable for biological sample detection and identification. Furthermore, the hybridization reactions can take up to two hours, which for many uses, such as detecting biological warfare agents, is simply too long. Therefore, a need exists for a device and system which can rapidly detect target molecules from samples.

The present invention is directed to achieving these objectives.

### SUMMARY OF THE INVENTION

The present invention relates to a method of manufacturing a detection device. The method first involves providing a substrate having a layer of aluminum between a first layer of photosensitive material and a base layer. Next, the substrate is subjected to a first level photolithography treatment to produce an aluminum electrical conductor containing conductive fingers with spaces between them. The spaces between the conductive fingers are covered with an electrical insulator material. Finally, biological probes are attached to the conductive fingers under conditions effective to form a gap between the biological probes on the spaced apart conductive fingers, where a target molecule, if present in a sample, can bind to a pair of the biological probes on the spaced apart conductive fingers. This bridges the gap between the biological probes, allowing detection of the target molecule.

Another aspect of the present invention relates to a method of manufacturing a detection device, which first involves providing a substrate having a base layer. Next, a first layer of electrical insulator material is deposited on one side of the base layer. A layer of aluminum is deposited on the first layer of electrical insulator material. Next, a first layer of photosensitive material is then coated onto the layer of aluminum. Certain portions of the first layer of photosensitive material are exposed to ultraviolet light through a first photomask, and the first layer of photosensitive material is developed and baked, leaving portions of the layer of aluminum uncovered. Then, the uncovered portions of the layer of aluminum are removed from the substrate, leaving portions of the first layer of electrical insulator material uncovered. Next, the photosensitive material remaining on the layer of aluminum is removed. Then, a diamond film is deposited on the substrate, and a second layer of photosensitive material is coated onto the diamond film. Next, the second layer of photosensitive material is exposed to ultraviolet light through a second photomask, and the second layer of photosensitive material is developed and baked, leaving portions of the diamond film uncovered. Then, the uncovered portions of the diamond film are removed from the layer of aluminum, where the exposing the second layer of photosensitive material, the developing and baking the second layer of photosensitive material, and the removing the uncovered portions of the diamond film are carried out such that only portions of the diamond film aligned with the conductive fingers will be removed, leaving portions of the second layer of photosensitive material on the substrate. Next, the second layer of photosensitive material remaining on the diamond film is removed. Finally, biological probes are attached to the conductive fingers under conditions effective to form a gap between the biological probes on the spaced apart conductive fingers, As a result, a target molecule, if present in a sample, can bind to a pair of the biological probes on the spaced apart conductive fingers to bridge the gap between the biological probes, allowing detection of the target molecule.

The present invention also relates to a method of manufacturing a detection device, which involves providing a substrate having an aluminum electrical conductor containing a plurality of coplanar conductive fingers with spaces between them, where the spaces are covered with an electrical insulator material. Biological probes are then attached to the conductive fingers under conditions effective to form a gap between probes on the spaced apart, coplanar, conductive fingers. As a result, a target molecule, if present in a sample, can bind to a pair of the biological probes on the spaced apart, coplanar, conductive fingers to bridge the gap between the biological probes, allowing detection of the target molecule.

Another aspect of the present invention relates to a method of manufacturing a detection device. The method first involves providing a substrate having a layer of aluminum and a first layer of photosensitive material. Then, the substrate is subjected to a first level photolithography treatment to produce an aluminum electrical conductor having conductive fingers with spaces between them. Finally, biological probes are attached to the conductive fingers under conditions effective to form a gap between the biological probes on the spaced apart conductive fingers. As a result, a target molecule, if present in a sample, can bind to a pair of the biological probes on the spaced apart conductive fingers to bridge the gap between the biological probes, allowing detection of the target molecule.

The present invention provides methods of fabricating a device for rapidly detecting the presence of biological material. The target molecule either itself or as a support is used to complete an electrical circuit. The presence of the target molecule is indicated by the ability to conduct an electrical signal through the circuit. In the case where the target molecule is not present, the circuit will not be completed. Thus, the target molecule acts as a switch. The presence of the target molecule provides an “on” signal for an electrical circuit, whereas the lack of the target molecule is interpreted as an “off” signal. Due to the direct detection of the target molecule, the device allows for extremely sensitive detection of target molecules connecting two electrical conductors.

### BRIEF DESCRIPTION OF THE DRAWINGS

FIGS. 1A-P illustrate the sequence of steps necessary for fabricating a device for detecting the presence of a target molecule. FIG. 1A depicts the cross sectional view of a substrate having a base layer. FIG. 1B is a cross sectional view of a substrate where a first layer of electrical insulator material has been deposited on one side of the base layer shown in FIG. 1A. FIG. 1C illustrates the cross sectional view of a substrate where a layer of aluminum has been deposited on the first layer of electrical insulator material shown in FIG. 1B. FIG. 1D depicts the cross sectional view of a substrate where a first layer of photosensitive material has been coated onto the layer of aluminum shown in FIG. 1C. FIG. 1E shows the cross sectional view of a substrate where certain portions of the first layer of photosensitive material shown in FIG. 1D are exposed to ultraviolet light through a first photomask. FIG. 1F is a cross sectional view of a substrate where the first layer of photosensitive material shown in FIG. 1E has been developed and baked, leaving portions of the layer of aluminum uncovered. FIG. 1G illustrates the cross sectional view of a substrate where the uncovered portions of the layer of aluminum shown in FIG. 1F has been removed, leaving portions of the first layer of electrical insulator material uncovered. FIG. 1H is a cross sectional view of a substrate where the photosensitive material remaining on the layer of aluminum shown in FIG. 1G has been removed. FIG. 1I shows the cross sectional view of a substrate where a diamond film has been deposited on the substrate shown in FIG. 1H. FIG. 1J illustrates the cross sectional view of a substrate where a second layer of photosensitive material has been coated onto the diamond film shown in FIG. 1I. FIG. 1K depicts the cross sectional view of a substrate where the second layer of photosensitive material shown in FIG. 1J is being exposed to ultraviolet light through a second photomask. FIG. 1L is a cross sectional view of a substrate where the second layer of photosensitive material shown in FIG. 1K has been developed and baked, leaving portions of the diamond film uncovered. FIG. 1M shows the cross sectional view of a substrate where the uncovered portions of the diamond film have been removed from the layer of aluminum, leaving portions of the second layer of photosensitive material on the substrate. FIG. 1N illustrates the cross sectional view of a substrate where the second layer of photosensitive material remaining on the diamond film shown in FIG. 1M has been removed. FIG. 1O depicts the top view of the fabricated device before the biological probes are attached. FIG. 1P shows the top view of the fabricated device after the biological probes are attached.

FIG. 2A illustrates an embodiment of the present invention where oligonucleotide probes are attached to the spaced part conductive fingers of the fabricated device shown in FIG. 1P. FIG. 2B shows how a target nucleic acid molecule present in a sample is detected by the fabricated device shown in FIG. 1P.

### DETAILED DESCRIPTION OF THE INVENTION

The present invention relates to a method of manufacturing a detection device. The method first involves providing a substrate having a layer of aluminum between a first layer of photosensitive material and a base layer. Next, the substrate is subjected to a first level photolithography treatment to produce an aluminum electrical conductor containing conductive fingers with spaces between them. The spaces between the conductive fingers are covered with an electrical insulator material. Finally, biological probes are attached to the conductive fingers under conditions effective to form a gap between the biological probes on the spaced apart conductive fingers, where a target molecule, if present in a sample, can bind to a pair of the biological probes on the spaced apart conductive fingers. This bridges the gap between the biological probes, allowing detection of the target molecule.

Another aspect of the present invention relates to a method of manufacturing a detection device, which first involves providing a substrate having a base layer. Next, a first layer of electrical insulator material is deposited on one side of the base layer. A layer of aluminum is deposited on the first layer of electrical insulator material. Next, a first layer of photosensitive material is then coated onto the layer of aluminum. Certain portions of the first layer of photosensitive material are exposed to ultraviolet light through a first photomask, and the first layer of photosensitive material is developed and baked, leaving portions of the layer of aluminum uncovered. Then, the uncovered portions of the layer of aluminum are removed from the substrate, leaving portions of the first layer of electrical insulator material uncovered. Next, the photosensitive material remaining on the layer of aluminum is removed. Then, a diamond film is deposited on the substrate, and a second layer of photosensitive material is coated onto the diamond film. Next, the second layer of photosensitive material is exposed to ultraviolet light through a second photomask, and the second layer of photosensitive material is developed and baked, leaving portions of the diamond film uncovered. Then, the uncovered portions of the diamond film are removed from the layer of aluminum, where the exposing the second layer of photosensitive material, the developing and baking the second layer of photosensitive material, and the removing the uncovered portions of the diamond film are carried out such that only portions of the diamond film aligned with the conductive fingers will be removed, leaving portions of the second layer of photosensitive material on the substrate. Next, the second layer of photosensitive material remaining on the diamond film is removed. Finally, biological probes are attached to the conductive fingers under conditions effective to form a gap between the biological probes on the spaced apart conductive fingers. As a result, a target molecule, if present in a sample, can bind to a pair of the biological probes on the spaced apart conductive fingers to bridge the gap between the biological probes, allowing detection of the target molecule.

The present invention also relates to a method of manufacturing a detection device, which involves providing a substrate having an aluminum electrical conductor containing a plurality of coplanar conductive fingers with spaces between them, where the spaces are covered with an electrical insulator material. Biological probes are then attached to the conductive fingers under conditions effective to form a gap between probes on the spaced apart, coplanar, conductive fingers. As a result, a target molecule, if present in a sample, can bind to a pair of the biological probes on the spaced apart, coplanar, conductive fingers to bridge the gap between the biological probes, allowing detection of the target molecule.

Another aspect of the present invention relates to a method of manufacturing a detection device. The method first involves providing a substrate having a layer of aluminum and a first layer of photosensitive material. Then, the substrate is subjected to a first level photolithography treatment to produce an aluminum electrical conductor having conductive fingers with spaces between them. Finally, biological probes are attached to the conductive fingers under conditions effective to form a gap between the biological probes on the spaced apart conductive fingers. As a result, a target molecule, if present in a sample, can bind to a pair of the biological probes on the spaced apart conductive fingers to bridge the gap between the biological probes, allowing detection of the target molecule.

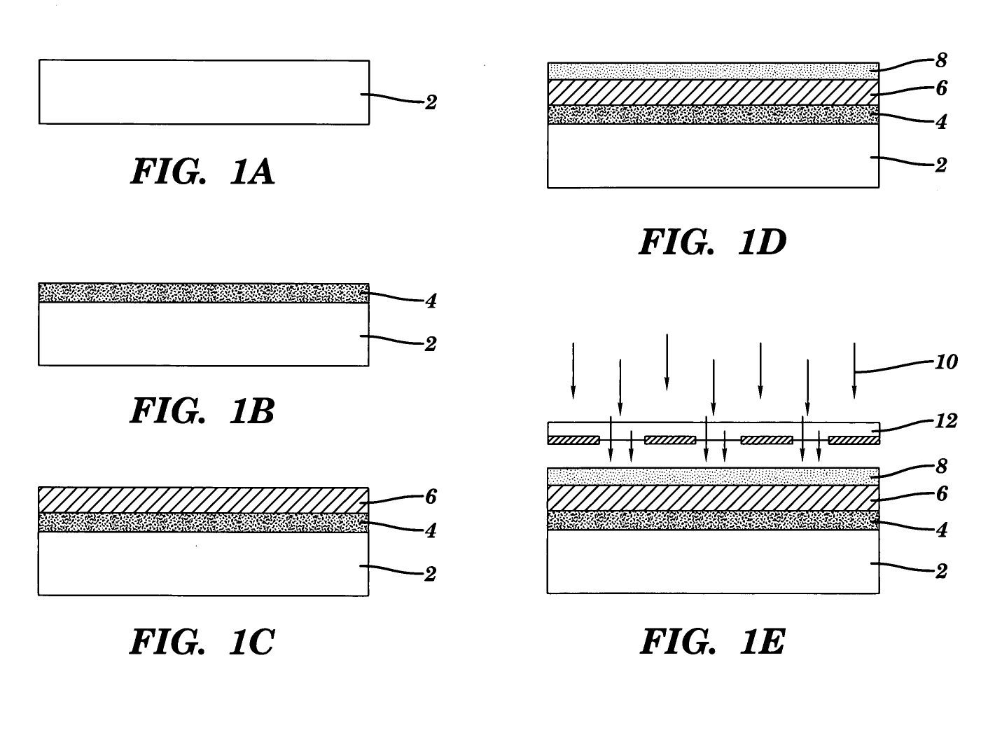

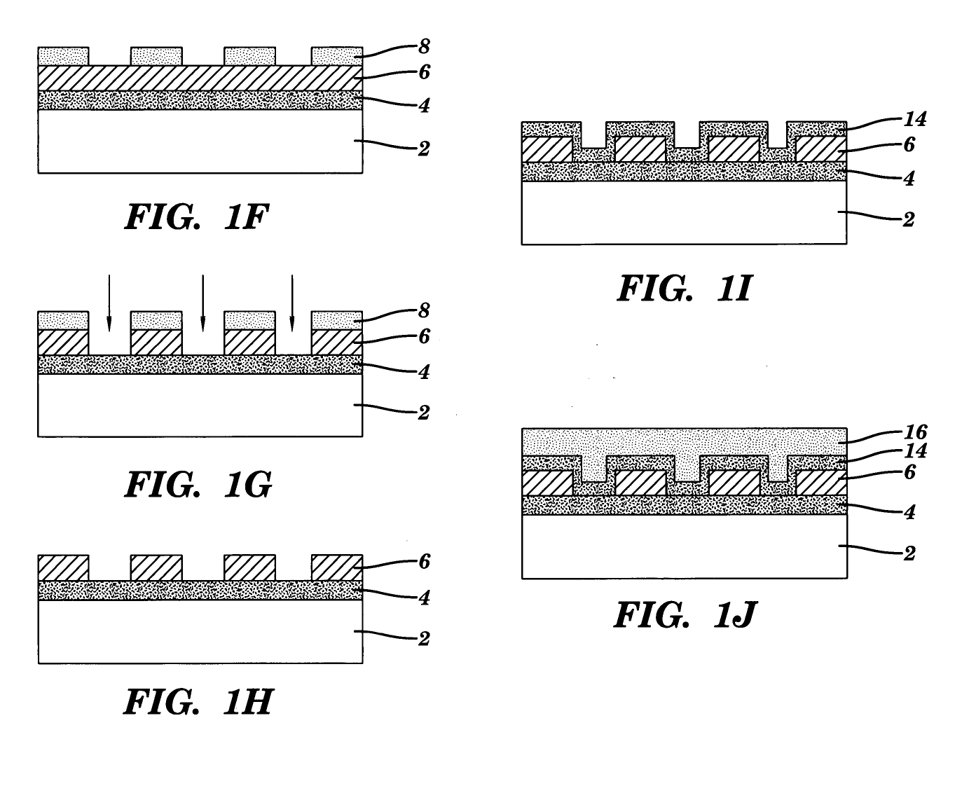

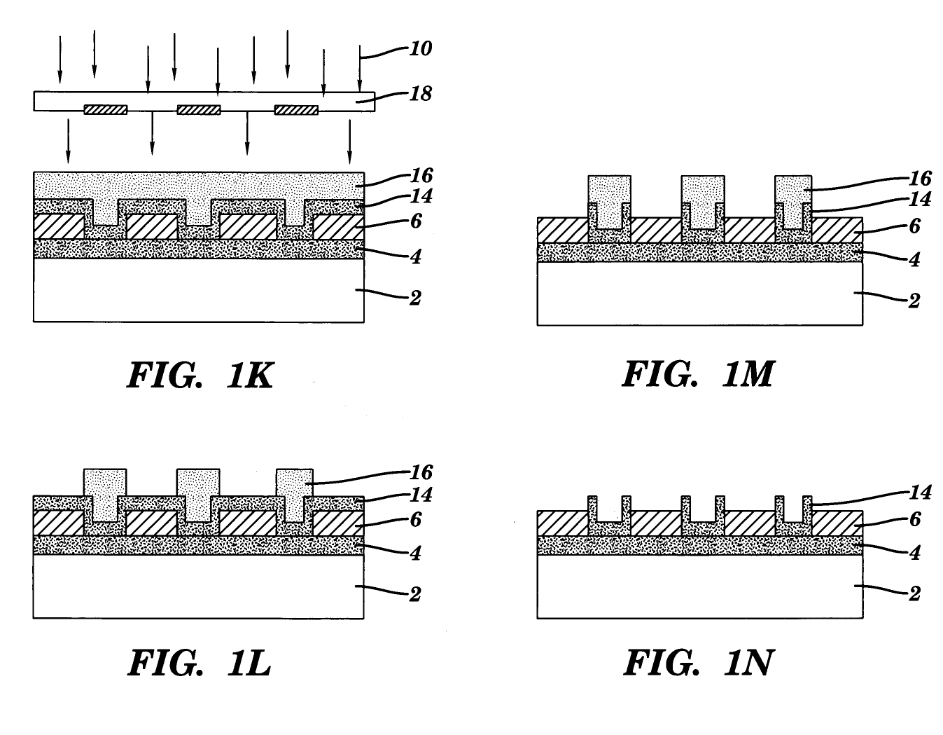

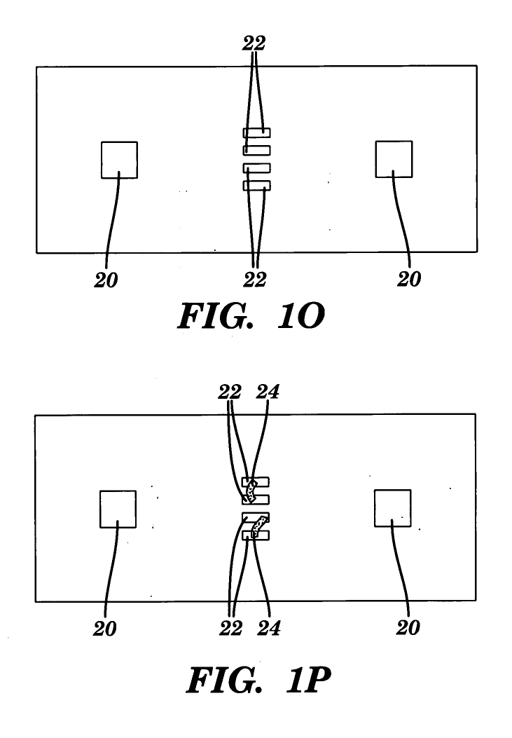

One embodiment of the method of the present invention is set forth in FIGS. 1A-P which depict the sequence of steps necessary for fabricating a device for detecting the presence of a target molecule.

This method first involves providing a substrate having base layer 2, as shown in FIG. 1A. An example of a suitable base layer material is silicon.

First layer of electrical insulator material 4 is deposited on one side of base layer 2, as shown in FIG. 1B. Examples of useful electrical insulator materials include silicon hard coat, silicon nitride, silicon dioxide, and polyimide.

Layer of aluminum 6, an electrically conductive material, is deposited on first layer of electrical insulator material 4, as shown in FIG. 1C.

First layer of photosensitive material 8 is coated onto layer of aluminum 6, as shown in FIG. 1D. Various photoresists, which are selected depending upon the exposing wavelength, can be used for this purpose.

Certain portions of first layer of photosensitive material 8 are exposed to ultraviolet light 10 through first photomask 12, as shown in FIG. 1E. Lithographic techniques used in the semiconductor manufacturing industries, such as photolithographic etching, plasma etching, or wet chemical etching, can be employed in the present invention. Alternatively, micromachining methods, such as laser drilling, micromilling and the like can be utilized.

First layer of photosensitive material 8 is developed and baked, leaving portions of layer of aluminum 6 uncovered, as shown in FIG. 1F. Such development and baling is carried out by developing the exposed substrate in a base developer and hard baking on a hot plate.

Uncovered portions of layer of aluminum 6 are removed from the substrate, leaving portions of first layer of electrical insulator material 4 uncovered, as shown in FIG. 1G. Layer of aluminum 6 is removed by etching the substrate with an aluminum etchant.

Photosensitive material 8 remaining on layer of aluminum 6 is removed, as shown in FIG. 1H. This step can be carried out by using acetone. In one embodiment, portions of the first layer of photosensitive material that were exposed to ultraviolet light are removed. Alternatively, this step can be carried out such that the portions of the first layer of photosensitive material that were not exposed to ultraviolet light are removed.

Next, diamond film 14, an electrical insulator material, is deposited on the substrate, as shown in FIG. 1I.

Second layer of photosensitive material 16 is coated onto the diamond film 14, as shown in FIG. 1J. Various photoresists, which are selected depending upon the exposing wavelength, can be used for this purpose.

Second layer of photosensitive material 16 is exposed to ultraviolet light 10 through a second photomask 18, as shown in FIG. 1K.

Exposed second layer of photosensitive material 16 is developed and baked, leaving portions of diamond film 14 uncovered, as shown in FIG. 1L. Such development and baking is carried out by developing the exposed substrate in a base developer and hard baking on a hot plate.

As shown in FIG. 1M, the uncovered portions of diamond film 14 are removed from layer of aluminum 6. This is carried out by plasma etching in O₂. Only portions of diamond film 14 aligned with the conductive fingers are removed. Portions of second layer of photosensitive material 16 are left on the substrate, as shown in FIG. 1M.

Second layer of photosensitive material remaining 16 on diamond film 14 is removed, as shown in FIG. 1N. This step can be carried out by using acetone. In one embodiment, portions of the second layer of photosensitive material that were exposed to ultraviolet light are removed. Alternatively, this step can be carried out such that the portions of the second layer of photosensitive material that were not exposed to ultraviolet light are removed.

The top view of the final fabricated device with two contact cuts 20 and an active area with spaced apart conductive fingers 22 exposed is shown in FIG. 10.

Finally, biological probes 24 are attached to conductive fingers 22 under conditions effective to form a gap between biological probes 24 on spaced apart conductive fingers 22, as shown in FIG. 1P. As a result, a target molecule, if present in a sample, can bind to a pair of the biological probes on the spaced apart conductive fingers to bridge the gap between the biological probes, allowing detection of the target molecule. Details on methods of attaching biological molecules to electrically conductive surfaces can be found in U.S. Provisional Patent Application Serial No. 60/310,937, filed on Aug. 8, 2001, which is hereby incorporated by reference in its entirety.

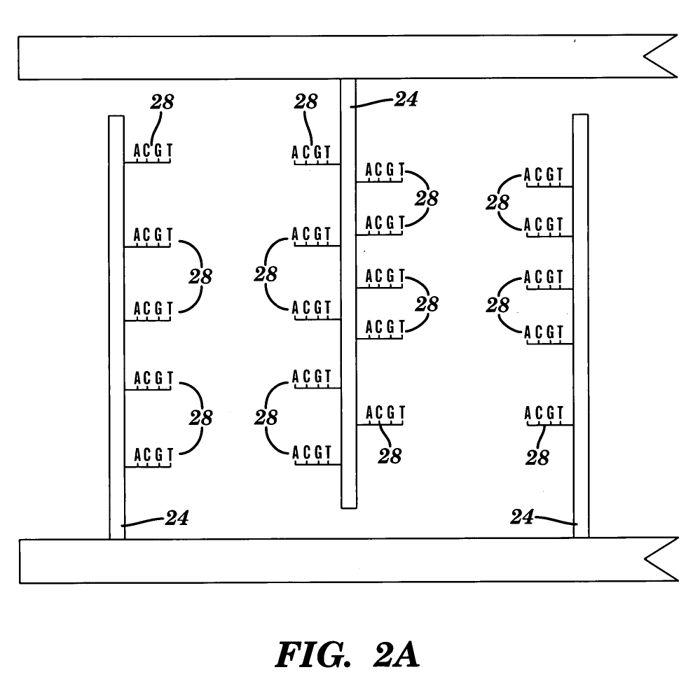

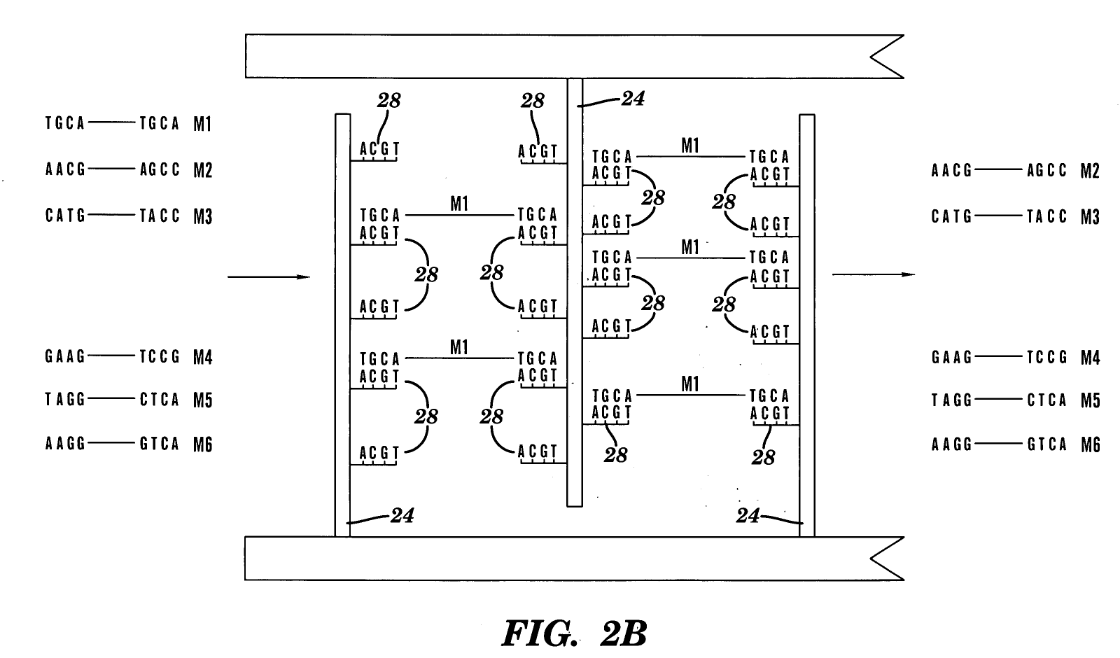

In one embodiment of the present invention, the biological probes are proteins or antibodies. FIG. 2 shows another embodiment of the present invention where the biological probes are oligonucleotide probes and the target molecule is a nucleic acid molecule. The oligonucleotide probes can be in the form of DNA, RNA, or protein nucleotide analogues. Such oligonucleotide probes are advantageously constructed from about 10 to 30 nucleotide bases. Shorter probe molecules have lower specificity for a target molecule, because there may exist in nature more than one target nucleic acid molecule with a sequence of nucleotides complementary to a shorter probe molecule. On the other hand, longer probe molecules have decreasingly small probabilities of complementary sequences with more than one natural target nucleic acid molecule. In addition, longer probe molecules exhibit longer hybridization times than shorter probe molecules. Since analysis time is a factor in a commercial device, the shortest possible probe that is sufficiently specific to the target nucleic acid molecule is desirable. Both the speed and specificity of binding target nucleic acid molecule to probe molecules can be increased if one electrical conductor has attached a probe molecule that is complementary to one end of the target nucleic acid molecule and the other electrical conductor has attached a probe that is complementary to the other end of the target nucleic acid molecule. In this case, even if short probe molecules that exhibit rapid hybridization rates are used, the specificity of the target molecule to the two probes is high.

The fabricated device of the present invention is used to detect target molecules from samples. As shown in FIG. 2A, oligonucleotide probes 26 attached to the spaced apart conductive fingers 22 are physically located at a distance sufficient that they cannot come into contact with one another. A sample, containing a mixture of nucleic acid molecules (i.e. M1-M6), to be tested is contacted with the fabricated device on which conductive fingers 22 are fixed, as shown in FIG. 2B. If a target nucleic acid molecule (i.e. M1) which is capable of binding to the two oligonucleotide probes is present in the sample, the target nucleic acid molecule can electrically connect the two probes. Any unhybridized nucleic acid molecules (i.e. M2-M6) not captured by the probes is washed away. Here, the electrical conductivity of nucleic acid molecules is relied upon to transmit the electrical signal. Hans-Werner Fink and Christian Schoenenberger reported in Nature (1999), which is hereby incorporated by reference in its entirety, that DNA conducts electricity like a semiconductor. This flow of current can be sufficient to construct a simple switch, which will indicate whether or not a target nucleic acid molecule is present within a sample. The presence of a target molecule can be detected as an “on” switch, while a set of probes not connected by a target molecule would be an “off” switch. The information can be processed by a digital computer which correlates the status of the switch with the presence of a particular target. The information can be quickly identified to the user as indicating the presence or absence of the biological material, organism, mutation, or other target of interest.

Optionally, after hybridization of the target molecules to sets of biological probes, the target molecule can be coated with a conductor, such as a metal, as described in U.S. patent application Ser. Nos. 60/095,096 or 60/099,506, which are hereby incorporated by reference in their entirety. The coated target molecule can then conduct electricity across the gap between the pair of probes, thus producing a detectable signal indicative of the presence of a target molecule.

### EXAMPLES

The following examples are provided to illustrate embodiments of the present invention but are by no means intended to limit its scope.

### Example 1

### Fabrication of a Detection Device with Aluminum Electrical Conductors

Fabrication began with a bare 6″ silicon wafer (n-type or p-type) with (100) orientation, a resistivity of 5-15 ohm·cm, and a thickness of 675+50 μm.

A layer of silicon hard coat, as a first electrical insulator layer, was spin-coated on one side of the silicon wafer. A puddle of silicon hard coat (methylsilsesquioxane solution; SHC1200, GE Silicones, Waterford, N.Y.), about 3 inches in diameter, was dispensed onto the wafer, which was then spun at 3000 rpm for 180 sec.

A layer of aluminum was either sputtered or evaporated to the desired thickness onto the layer of silicon hard coat. When sputtered, power was set at 2000 Watts, argon flow at 63 sccm, presputter time at 5 min, chamber pressure at 106 Torr, deposition pressure at 5 mTorr, and deposition rate at 240 A/min. Alternatively, an aluminum pellet was evaporated using the evaporator (CVC, Port Townsend, Wash.) set at an appropriate chamber pressure (low 10⁻⁶ Torr) to give an approximate thickness of 2000 Å.

A first layer of photoresist was coated onto the aluminum layer. The wafer was coated using coat trac or was hand-coated using Oir 620, a positive photoresist containing novalac resin, diazonaphthoquinone, casting solvent, additives, and surfactants (Olin, Norwalk, Conn.). When hand-coating, the substrate must be dehydration baked at 200° C. for 2 min on a hot plate, the photoresist must be dispensed at 4500 rpm for 1 min, and the substrate must be soft baked at 90° C. for 1 min on a hot plate.

The substrate was subjected to a first level lithography treatment, using an i-line stepper (Canon, Japan). The stepper job was loaded using the command, “ST DNA3_level 1.” The exposure dose was set at 130 mj/cm².

The exposed wafer was developed in a base developer (Shipley CD26, Shipley, Marboro, Mass.) for 1 min and rinsed with water. The wafer was dried and hard baked on a hot plate at 120° C. for 2 min.

The exposed aluminum was removed by etching the wafer with aluminum etchant at 50° C., and rinsing well in DI water. The etching time depended on the thickness of aluminum, and the etch rate was 2000 Å/min.

The photoresist remaining on the aluminum layer was removed with acetone and rinsed with DI water.

A diamond film, as a second electrical insulator layer, was spin-coated onto the substrate.

The substrate was then coated with photoresist as previously described.

The substrate was subjected to a second level lithography treatment to open active areas and contact cuts, using an i-line stepper (Canon, Japan). The stepper job was loaded using the command, “ST DNA3_level 1.” The substrate was exposed and a post-exposure bake was performed as described for the first level lithography. An exposure dose of 130 mj/cm² was used.

The exposed substrate was developed in the base developer for 1 min and rinsed with water. Then, the substrate was dried and hard baked on a hot plate at 120° C. for 2 min.

Next, the exposed diamond film was removed by plasma etching in O₂. This step is not necessary if photoresist is used as the second electrical insulator layer.

Next, the photoresist remaining on the aluminum layer was removed with acetone. This step is not necessary if photoresist is used as the second electrical insulator layer.

Finally, biological probe molecules are attached to the active area of the fabricated device with the aluminum electrical conductors exposed.

Although the invention has been described in detail for the purpose of illustration, it is understood that such detail is solely for that purpose, and variations can be made therein by those skilled in the art without departing from the spirit and scope of the invention which is defined by the following claims.

1. A method of manufacturing a detection device, said method comprising:
providing a substrate having a layer of aluminum between a first layer of photosensitive material and a base layer;
subjecting the substrate to a first level photolithography treatment to produce an aluminum electrical conductor comprising conductive fingers with spaces between them;
covering the spaces between the conductive fingers with an electrical insulator material; and
attaching biological probes to the conductive fingers under conditions effective to form a gap between the biological probes on the spaced apart conductive fingers, whereby a target molecule, if present in a sample, can bind to a pair of the biological probes on the spaced apart conductive fingers to bridge the gap between the biological probes, allowing detection of the target molecule.

2. A method according to claim 1, wherein the biological probes are oligonucleotide probes and the target molecule is a nucleic acid molecule.

3. A method according to claim 1, wherein the biological probes are proteins or antibodies.

4. A method according to claim 1, wherein said substrate further comprises a first layer of electrical insulator material between the base layer and the layer of aluminum.

5. A method according to claim 4, wherein said providing a substrate comprises:
depositing the first layer of electrical insulator material on one side of the base layer;
depositing the layer of aluminum on the first layer of electrical insulator material; and
coating the first layer of photosensitive material onto the layer of aluminum.

6. A method according to claim 4, wherein said subjecting the substrate to first level photolithography treatment comprises:
exposing certain portions of the first layer of photosensitive material to ultraviolet light through a first photomask;
developing and baking the first layer of photosensitive material leaving portions of the layer of aluminum uncovered;
removing the uncovered portions of the layer of aluminum from said substrate, leaving portions of the first layer of electrical insulator material uncovered; and
removing the photosensitive material remaining on the layer of aluminum.

7. A method according to claim 6, wherein said method is carried out such that the portions of the first layer of photosensitive material that were exposed to ultraviolet light are removed.

8. A method according to claim 6, wherein said method is carried out such that the portions of the first layer of photosensitive material that were not exposed to ultraviolet light are removed.

9. A method according to claim 1, wherein said covering the spaces between the conductive fingers with an electrical insulator material comprises:
depositing a diamond film on said substrate after said subjecting the substrate to a first level photolithography treatment;
coating a second layer of photosensitive material onto the diamond film; and
subjecting the substrate to a second level photolithography treatment to cover the spaces between the conductor fingers with the diamond film.

10. A method according to claim 9, wherein said subjecting the substrate to a second level photolithography treatment comprises:
exposing the second layer of photosensitive material to ultraviolet light through a second photomask;
developing and baking the second layer of photosensitive material, leaving portions of the diamond film uncovered;
removing the uncovered portions of the diamond film from said layer of aluminum, wherein said exposing, said developing and baking, and said removing the uncovered portions of the diamond film during said second level photolithography treatment are carried out such that only portions of the diamond film aligned with the conductive fingers will be removed, leaving portions of the second layer of photosensitive material on the substrate; and
removing the second layer of photosensitive material remaining on the diamond film.

11. A method according to claim 10, wherein said method is carried out such that the portions of the second layer of photosensitive material that were exposed to ultraviolet light are removed.

12. A method according to claim 10, wherein said method is carried out such that the portions of the second layer of photosensitive material that were not exposed to ultraviolet light are removed.

13. A method according to claim 1, wherein the electrical insulator material covering the spaces between the conductive fingers is a diamond film.

14. A method of manufacturing a detection device, said method comprising:
providing a substrate having a base layer,
depositing a first layer of electrical insulator material on one side of the base layer;
depositing a layer of aluminum on the first layer of electrical insulator material;
coating a first layer of photosensitive material onto the layer of aluminum;
exposing certain portions of the first layer of photosensitive material to ultraviolet light through a first photomask;
developing and baking the first layer of photosensitive material, leaving portions of the layer of aluminum uncovered;
removing the uncovered portions of the layer of aluminum from said substrate, leaving portions of the first layer of electrical insulator material uncovered;
removing the photosensitive material remaining on the layer of aluminum;
depositing a diamond film on said substrate;
coating a second layer of photosensitive material onto the diamond film;
exposing the second layer of photosensitive material to ultraviolet light through a second photomask;
developing and baking the second layer of photosensitive material, leaving portions of the diamond film uncovered;
removing the uncovered portions of the diamond film from said layer of aluminum, wherein said exposing the second layer of photosensitive material, said developing and baking the second layer of photosensitive material, and said removing the uncovered portions of the diamond film are carried out such that only portions of the diamond film aligned with the conductive fingers will be removed, leaving portions of the second layer of photosensitive material on the substrate; and
removing the second layer of photosensitive material remaining on the diamond film; and
attaching biological probes to the conductive fingers under conditions effective to form a gap between the biological probes on the spaced apart conductive fingers, whereby a target molecule, if present in a sample, can bind to a pair of the biological probes on the spaced apart conductive fingers to bridge the gap between the biological probes, allowing detection of the target molecule.

15. A method according to claim 14, wherein the biological probes are oligonucleotide probes and the target molecule is a nucleic acid molecule.

16. A method according to claim 14, wherein the biological probes are proteins or antibodies.

17. A method according to claim 14, wherein said method is carried out such that the portions of the first layer of photosensitive material that were exposed to ultraviolet light are removed.

18. A method according to claim 14, wherein said method is carried out such that the portions of the first layer of photosensitive material that were not exposed to ultraviolet light are removed.

19. A method according to claim 14, wherein said method is carried out such that the portions of the second layer of photosensitive material that were exposed to ultraviolet light are removed.

20. A method according to claim 14, wherein said method is carried out such that the portions of the second layer of photosensitive material that were not exposed to ultraviolet light are removed.

21. A method of manufacturing a detection device, said method comprising:
providing a substrate having an aluminum electrical conductor comprising a plurality of coplanar conductive fingers with spaces between them, wherein the spaces are covered with an electrical insulator material; and
attaching biological probes to the conductive fingers under conditions effective to form a gap between probes on the spaced apart, coplanar, conductive fingers, whereby a target molecule, if present in a sample, can bind to a pair of the biological probes on the spaced apart, coplanar, conductive fingers to bridge the gap between the biological probes, allowing detection of the target molecule.

22. A method according to claim 21, wherein the biological probes are oligonucleotide probes and the target molecule is a nucleic acid molecule.

23. A method according to claim 21, wherein the biological probes are proteins or antibodies.

24. A method according to claim 21, wherein the electrical insulator material covering the spaces between the conductive fingers is a diamond film.

25. A method of manufacturing a detection device, said method comprising:
providing a substrate having a layer of aluminum and a first layer of photosensitive material;
subjecting the substrate to a first level photolithography treatment to produce an aluminum electrical conductor comprising conductive fingers with spaces between them; and
attaching biological probes to the conductive fingers under conditions effective to form a gap between the biological probes on the spaced apart conductive fingers, whereby a target molecule, if present in a sample, can bind to a pair of the biological probes on the spaced apart conductive fingers to bridge the gap between the biological probes, allowing detection of the target molecule.

26. A method according to claim 25, wherein said substrate further comprises a base layer wherein said layer of aluminum is between said first layer of photosensitive material and said base layer.

27. A method according to claim 25, further comprising:
covering the spaces between the conductive fingers with an electrical insulator material, prior to said attaching biological probes and after said subjecting the substrate to a first level photolithography.

28. A method according to claim 27, wherein the electrical insulator material covering the spaces between the conductive fingers is a diamond film.

29. A method according to claim 25, wherein the biological probes are oligonucleotide probes and the target molecule is a nucleic acid molecule.

30. A method according to claim 25, wherein the biological probes are proteins or antibodies.
